(19) United States
(12) Patent Application Publication
(10) Pub. No.: US 2012/0253333 A1
(43) Pub. Date: Oct. 4, 2012

(54) Combination Laser Treatment of Skin Conditions

(76) Inventors: JEROME M. GARDEN, Chicago, IL (US); ABNOEAL D. BAKUS, Chicago, IL (US); DINA YAGHMAI, Chicago, IL (US)

(21) Appl. No.: 13/305,764

(22) Filed: Nov. 29, 2011

Related U.S. Application Data
(60) Provisional application No. 61/470,883, filed on Apr. 1, 2011.

(51) Int. Cl. A61B 18/20

(52) U.S. Cl. 606/9

(57) ABSTRACT

A method and apparatus for treatment of health and skin conditions through the use of a combination of laser systems or a laser system capable of delivering different pulse durations. The laser system being capable of producing both Q-switched short pulsed laser energy and millisecond long pulsed laser energies. Multiple skin conditions would be susceptible to this treatment, including acne, skin aging and textural changes, pigmentary changes, scarring, rosacea, striae, redness and hair removal.

### CROSS-REFERENCE TO RELATED APPLICATIONS

This application claims priority to U.S. Provisional Patent Application No. 61/470,883, filed on Apr. 1, 2011, entitled “Combination Laser Treatment Of Skin Conditions,” by Jerome M. Garden et al., which is hereby incorporated by reference.

### TECHNICAL FIELD

The present disclosure relates to a system and method for the treatment of health and skin conditions. More specifically, the present invention relates to a system and method for laser treatment of health and skin conditions such as wrinkles, skin texturing (e.g., rough skin), excessively large pore-size, rosacea, blush/diffuse redness, striae (e.g., stretch marks), scarring, acne, excessive sebum (oil) production, sebaceous hyperplasia (enlarged oil glands), unwanted hair, kidney stones, gallbladder stones, and vessel plaque.

### BACKGROUND

Nearly every individual has been afflicted with one or more undesirable skin conditions. These skin conditions include, for example, wrinkles, undesirable skin texturing (e.g., rough skin), increased pore-size, rosacea, blush/diffuse redness, striae (e.g., stretch marks), scarring, acne, excessive sebum (oil) production, sebaceous hyperplasia (enlarged oil glands) and unwanted hair.

Acne vulgaris, more commonly referred to as simply “acne,” is one of the most common skin conditions and typically affects people from ages 10 through 40. Acne appears in a number of forms, including congested pores (“comedones”), either whiteheads or blackheads, pimples, pustules, or cysts (deep pimples, boils).

Acne often occurs when oil (sebaceous) glands become active, usually around puberty, and are stimulated by hormones in both boys and girls. While sebum (oil) is a natural substance used to lubricate and protect the skin, under certain circumstances, cells that are close to the surface may block the openings of the sebaceous glands causing a buildup of oil underneath. This oil buildup stimulates bacteria (which live on the skin and generally do not cause problems) multiplication and causes surrounding tissue to become inflamed—often leading to lesions. Inflammation near the skin's surface produces a pustule; deeper inflammation results in a papule (i.e., pimple); deeper still, a cyst. “Whiteheads” also may occur. If the material accumulates melanin pigment or becomes oxidized, the color changes from white to black, and the result is a “blackhead.”

After an acne lesion has healed, it can leave a red or hyperpigmented mark on the skin. This is actually not a scar, but rather a post-inflammatory change. The redness, or hyperpigmentation, is seen as the skin goes through its healing and remodeling process, which may take approximately 3-12 months before the skin can heal normally. At times, the acne lesions may also heal with loss of tissue or a raised scar. These effects can be permanent. In spite of significant advances in the treatment of acne, acne remains a very difficult condition to treat.

Acne is not the sole cause of persistent redness. Rosacea, often confused with classical acne, is characterized by pimples in the middle third of the face, along with redness, flushing, and superficial blood vessels.

Another cause of redness is pseudofolliculitis, which is also referred to as “razor bumps” or “razor rash.” Pseudofolliculitis can often occur after shaving when hairs are cut too close to the skin. As a result, hairs bend under the skin and produce pimples, which can lead to pigmentary changes and scarring. There are countless other acquired and genetic conditions which can lead to “permanent defects” or “scars” (e.g., folliculitis, gram-negative folliculitis, etc.). Regardless of the process by which they occur, “permanent defects” or “scars” are highly undesirable.

In addition, the changes that may naturally occur with aging (e.g., skin-texture, irregularities, wrinkling, skin laxity, pore-size increase, etc.) need an effective and noninvasive, therapeutic treatment.

Therefore, there is a need for an effective and noninvasive procedure or treatment that can reduce or eliminate aging changes, permanent defects, discolorations, and/or other scarring. Further, there exists a need for an effective and noninvasive procedure or treatment which can prevent the formation of acne and remove undesired hair, stretch marks and oil gland related issues.

### SUMMARY OF THE INVENTION

There is a need for an effective, noninvasive procedure to reduce or eliminate permanent defects, discolorations, and/or other scarring, age-related changes, striae, redness, excessive sebum (oil) production, sebaceous hyperplasia (enlarged oil glands), acne, large pore size, and unwanted hair. More particularly, there is a need for a laser whose wavelength may be exposed at multiple exposure times, thereby yielding advantageous results.

According to a first aspect of the present invention, a method for treating a condition, comprises: administering a Q-switched short pulsed ND:YAG laser energy having a pulse duration between 5 and 350 nanoseconds; and administering a millisecond long pulsed ND:YAG laser energy having a pulse duration between 1 and 100 milliseconds; wherein the Q-switched short pulsed laser energy and millisecond long pulsed laser energy are both applied to treat a health or age related condition.

According to a second aspect of the present invention, a method for treating a skin condition, comprises: administering a Q-switched short pulsed laser energy; and administering a millisecond long pulsed laser energy; wherein the Q-switched short pulsed laser energy and millisecond long pulsed laser energy are both applied to treat a skin condition.

According to a third aspect of the present invention, a laser system for treating one or more conditions, comprises: a power supply device; a lasing medium; one or more lenses; and a Q-switched apparatus for outputting Q-switched short pulsed laser energy; wherein the laser system is configured to output both millisecond long pulsed laser energy having a pulse duration between 0.1 and 500 milliseconds and Q-switched short pulsed laser energy having a pulse duration between 1 femtosecond and 400 nanoseconds.

According to a fourth aspect of the present invention, a laser system for treating one or more conditions, comprises: a power supply device; a lasing medium; one or more lenses; and a Q-switched apparatus for outputting Q-switched short pulsed laser energy; wherein the laser system is configured to output both millisecond long pulsed laser energy having a pulse duration between 0.1 and 500 milliseconds and Q-switched short pulsed laser energy having a pulse duration between 1 femtosecond and 400 nanoseconds.

According to a fifth aspect of the present invention, laser apparatus for treating one or more skin conditions comprises: a power supply device; a lasing medium; one or more lenses; and a Q-switched apparatus for outputting Q-switched short pulsed laser energy; wherein the laser system is used to treat a skin condition and is configured to output both millisecond long pulsed laser energy having a pulse duration between 0.1 and 500 milliseconds and Q-switched short pulsed laser energy having a pulse duration between 1 femtosecond and 400 nanoseconds.

In some aspects, the Q-switched, short-pulsed-laser energy and millisecond-long, pulsed-laser energy may be delivered successively or simultaneously.

In other aspects, the Q-switched, short-pulsed-laser energy and millisecond-long, pulsed-laser energy may be delivered using a single laser system or a laser system enabled to produce both Q-switched, short-pulse energies and millisecond-long pulses.

In certain aspects, the millisecond-long, pulsed-laser energy may have a pulse duration between 0.1 and 500 milliseconds and the Q-switched short-pulse may have a pulse duration between 1 femtosecond and 400 nanoseconds. In other aspects, the millisecond-long, pulsed-laser energy may have a pulse duration between 1 and 100 milliseconds and the Q-switched short-pulse may have a pulse duration between 5 to 350 nanoseconds.

In another aspect, the millisecond-long, pulsed-laser energy and Q-switched, short-pulsed-laser energy may be generated using a ruby crystal as a gain medium, a Nd:YAG lasing medium, a lasing medium chosen from Table 1, or another lasing medium that yields a desirable wavelength.

In certain aspects, a laser system, method, or apparatus enabled to produce both Q-switched, short-pulse energies and millisecond-long pulses may be used to treat inflammatory skin conditions, acne, skin aging, skin laxity, skin-texture irregularities, enlarged pores, scars, pigmentary changes, changes in skin color and luster, skin growths, cancers, excessive sebum (oil) production, sebaceous hyperplasia (enlarged oil glands) and unwanted hair and/or tattoos. Similarly, the laser system, method, or apparatus may also be enabled to treat kidney stones, gallbladder stones, vessel plaque, and combinations thereof.

### DESCRIPTION OF THE DRAWINGS

These and other advantages of the present invention will be readily understood with reference to the following specification and attached drawings wherein:

FIG. 1 is a high-level exemplary diagram of a first embodiment of a laser enabled to emit both millisecond-long and Q-switched short pulses;

FIG. 2a is a high-level exemplary diagram of a second embodiment of a laser enabled to emit both millisecond-long and Q-switched short pulses;

FIG. 2b is a variation of FIG. 2a where two separate power/trigger supplies are used to deliver millisecond-long and Q-switched short pulses;

FIG. 3a is a high-level exemplary diagram of a third embodiment of a laser enabled to emit both millisecond-long and Q-switched short pulses;

FIG. 3b is a high-level exemplary diagram of the third embodiment of FIG. 3a wherein a beam splitter is used;

FIG. 3c is a high-level exemplary diagram of the third embodiment of FIG. 3b wherein a single beam delivery system is used;

FIG. 4a is a bar graph illustrating the percentages of acne lesional reduction after treatment using a laser system of the present invention; and

FIG. 4b is a bar graph illustrating the overall improvement of skin appearance after treatment using a laser system of the present invention.

### DETAILED DESCRIPTION

Preferred embodiments of the present invention will be described hereinbelow with reference to the accompanying drawings. In the following description, well-known functions or constructions are not described at length because they may tend to obscure the invention in unnecessary detail.

There are a number of methods for treating and/or removing “permanent defects” or “scars.” One of the oldest and most widely used methods is dermabrasion, which may be roughly divided into two categories—dermabrasion and microdermabrasion. Although both procedures mechanically remove the surface of the skin by abrasive techniques (e.g., sanding), a key difference between dermabrasion and microdermabrasion is that microdermabrasion peels off only the top 10-15 microns (a very fine layer), whereas dermabrasion exfoliates much more deeply. Dermabrasion is far more effective but also more invasive (i.e., more painful with an initially less attractive raw appearance) and can take months to heal.

Exfoliation with microdermabrasion is more gentle and less invasive. Not surprisingly, microdermabrasion is not as effective on deep wrinkles and scars because it removes only a very fine layer of skin, but may be useful in diminishing fine lines, superficial scars, age spots, and sunspots, while also helping with acne.

More recently, the laser has proven to be a versatile tool in the treatment of various skin conditions. Lasers remove defects by ablating the top layers of skin to a precise, predetermined depth. As in dermabrasion techniques, the area heals by replacing the ablated skin layers with skin having a newer appearance. Lasers may also be used in a non-ablative manner to stimulate the skin to correct texture changes, scarring, and pigmentary changes. Lasing mediums suitable for skin treatment, such as the system and method disclosed herewith, include, for example, those listed in Table 1.

| Lasing | |
|---|---|
| medium | Approximate Wavelength (nm) |
| Argon | 488-514 |
| Intense Pulse | 500-1200 |

TABLE 1

| Light (IPL) | |
|---|---|
| Dye | 540/570-640 |
| Copper | 510/578 |
| Gold Vapor | 627 |
| Krypton | 416/531/568/752/800 |
| KTP/Diode | 532 |
| Diode | 800/940/980/1450 |
| Nd: YAG | 1,064/1,320/1,440/1,550 |
| Nd: YVO₄ | 1,064 |
| Nd: YLF | 1,047/1,053 |
| Er: YAG | 1,550/2,940 |
| Er: glass | 1,540 |
| Thulium | 1,927 |
| Er: YSGG | 2,780 |
| Holmium | 2,100 |
| CO₂ | 10,600 |
| Ruby | 694 |
| Alexandrite | 755 |

The use of lasers on acne is not entirely new. A first method of treating skin conditions, including acne, is disclosed in U.S. Pat. No. 5,817,089 to Tankovich, et al. Tankovich discloses the treatment of skin conditions by removing the top layer of skin using short Nd:YAG laser pulses. Other prior attempts have included application of a topical carbon-based material to enhance laser absorption and remove surface skin. This is more commonly referred to as the “laser peel method.”

A second method of treating acne is disclosed in U.S. Pat. No. 7,703,458 to Levernier, et al. Levernier discloses the treatment of acne, wrinkles, unwanted hair, leg veins, and a number of other dermatologic conditions by using a pulsed, high-peak power laser (Nd:YAG).

An example combination laser is disclosed by U.S. Patent Pub. No. 2008/03119431 entitled “Method Of Curing Inflammatory Acne By Using Carbon Lotion And Pulsed Laser” to Hwang et al (the “Hwang reference”). The Hwang reference discloses a method to assist in curing inflammatory acne by applying a carbon lotion onto an area covered with acne, irradiating the applied carbon lotion with a laser pulse having a pulse width of microsecond, and irradiating the applied carbon lotion with a laser pulse having a pulse width of nanosecond to sterilize acne bacilli and open skin pores clogged with sebum, thereby entirely treating the inflammatory acne. The method requires the application of a carbon lotion onto an epidermis to be cured, irradiating the carbon lotion with a laser pulse to heat and burst the applied carbon lotion. However, contrary to the Hwang reference, the present application does not, for example, require the use, or application, of a cream nor is the laser system of the present application restricted to nanosecond and microsecond pulses. For instance, the combination laser disclosed by the present application may be enabled to yield longer pulses ranging from, for example, less than a millisecond to more than 500 milliseconds, while Q-switched laser pulses may range from a femtosecond to many nanoseconds.

Laser treatment and laser surgery have been in use for some time to reduce the scars left behind by acne, but recent research suggests that laser treatment may also prevent the formation of acne. This may be due in part to the laser's ability to reduce the formation of bacteria and to diminish oil-producing sebaceous glands.

Another method of treating acne is disclosed in U.S. Pat. No. 6,235,016 to Stewart, hereby incorporated by reference in its entirety herein. Stewart discloses a method of reducing acne-causing sebum production in human skin by using pulsed light having a wavelength, or a wavelength range, that targets the lipids contained within the sebaceous gland, thus destroying the differentiated and mature sebocytes by photothermolysis without damaging either the surrounding and overlying tissue or the basement membrane of the sebaceous gland.

As discussed above, using lasers to treat various conditions has been well-documented and accepted as a standard of care; however, limitations of outcome and associated side effects have made an improved therapeutic approach still desirable. Treatment entails the use of a laser wavelength and exposure time to target a tissue chromophore to effect a desired outcome. An objective is to choose a laser that has an effective and/or selective wavelength emission in addition to a particular pulse duration. Selection of a certain wavelength allows for light interaction with the desired target, thus reducing collateral thermal damage to the surrounding tissue. The wavelength may be chosen by selecting a lasing medium that yields a desirable wavelength.

Popular laser types used for resurfacing acne scars include, for example, carbon dioxide (“CO₂”) and Erbium:Yttrium Aluminum Garnet lasers (“Er:YAG”).

Lasers that emit energy in a continuous exposure are limited in that they are controlled only by turning the laser off or by blocking emitted light being released to their target. Older laser technologies, such as the continuous wave (“CW”) CO₂ (10,600 nm peak), are largely being replaced with quasi-CW mode lasers, pulsed laser systems and fractional delivery laser systems.

Er:YAG lasers are solid-state lasers whose lasing medium is erbium-doped yttrium aluminum garnet (Er:Y3Al5O12). Er:YAG lasers typically emit light with an infrared wavelength of 2940 nm. Unlike the Nd:YAG lasers, the frequency of Er:YAG lasers may be strongly absorbed by water because of atomic resonances. This limits the use of Er:YAG lasers in surgery and many other laser applications where water may be present, but it can be useful in laser skin resurfacing and fractional lasers.

Nd:YAG lasers are a type of solid-state laser that use a crystal as a lasing medium and may be optically pumped using a flashtube or laser diodes. The triply-ionized dopant neodymium typically replaces the similar-sized yttrium in the crystal structure of the yttrium aluminum garnet (YAG). Nd:YAG lasers can typically emit infrared light with a wavelength of 1064 nm and with transitions near 940, 1120, 1320, and 1440 nm.

To adjust pulse durations, a pulsed Nd:YAG laser may be operated in a Q-switched mode. A Q-switched laser may apply active and/or passive Q-switching techniques, allowing the laser to emit energetic pulses. Q-switching, which is discussed in greater detail below, may be accomplished by inserting an optical “switch” in the laser cavity that, for example, waits for a maximum population inversion in the neodymium ions before it opens, providing output powers of up to 250 megawatts and pulse durations of 5 to 25 nanoseconds. The high-intensity pulses may be efficiently frequency-doubled to generate laser light at 532 nm or higher harmonics at 355 and 266 nm.

In a Q-switched laser, the population inversion permits build-up by introducing loss inside the resonator that exceeds the gain of the medium; this can also be described as a reduction of the quality factor or “Q” of the cavity. Then, once the pump energy stored in the laser medium has approached the maximum possible level, the introduced loss mechanism occur (often an electro- or acousto-optical element) may be rapidly removed (or by itself in a passive device), allowing lasing to begin, which rapidly obtains the stored energy in the gain medium. This results in a short pulse incorporating that energy and thus a high peak power.

A mode-locked laser may be enabled to emit extremely short pulses ranging from tens of picoseconds down to less than ten femtoseconds. These pulses may repeat at the round trip time, that is, the time that it takes light to complete one round trip between the mirrors comprising the resonator. Due to the Fourier limit (also known as energy-time uncertainty), a pulse of such short temporal length has a spectrum spread over a considerable bandwidth. Thus such a gain medium must have a gain bandwidth sufficiently broad to amplify those frequencies. An example of a suitable material may be titanium-doped, artificially grown sapphire (Ti:sapphire), which has a very wide gain bandwidth and can thus produce pulses of only a few femtoseconds duration.

Such mode-locked lasers are a versatile tool for researching processes occurring on extremely short time scales (known as femtosecond physics, femtosecond chemistry, and ultrafast science), for maximizing the effect of nonlinearity in optical materials (e.g., in second-harmonic generation, parametric down-conversion, optical parametric oscillators, and the like) due to the large peak power, and in ablation applications and tattoo removal. Again, because of the extremely short pulse duration, such a laser may produce pulses that achieve an extremely high peak power.

Another method of achieving pulsed-laser operation may be to pump the laser material with a source that may be itself pulsed, either through electronic charging in the case of flash lamps, or through another laser which may be already pulsed. Pulsed pumping was historically used with dye lasers where the inverted population lifetime of a dye molecule was so short that a high-energy, fast pump was needed. A way to overcome this problem may be to charge large capacitors which are then switched to discharge through flash lamps, producing an intense flash. Pulsed pumping is often used for three-level lasers in which the lower energy level rapidly becomes highly populated, preventing further lasing until those atoms relax to the ground state.

Ruby crystal lasers were the earliest Q-switched laser on the market and emit light at 694 nm. Unfortunately, ruby crystal lasers are often disadvantaged by certain inherent characteristics of the ruby; for example, it cannot fire as quickly as similarly powered YAG lasers. Whereas a YAG laser can be fired as fast as 10 Hz (10 shots/second), a ruby can be fired only up to 2 Hz.

Alexandrite lasers emit light at 755 nm, which may be a suitable wavelength for the removal of tattoos and pigmented lesions. As a non-Q-switched laser, it may also be used for hair removal. However, numerous lasing mediums, including those that may be Q-switched, can be used for hair removal. For example, recent evidence exhibited the benefit of using QS:YAG laser in treating fine-caliber pigmented hairs. For many patients, single laser approaches to hair removal have not been fully effective, because the use of a given laser may result in the reduction of only hair shafts with a specific diameter or in the miniaturization of the hair shaft, resulting in the persistence of many hairs.

Therefore, a study was executed to evaluate the effectiveness of sequential treatments with two different lasers (i.e., Q-switched and millisecond long) for improved hair removal and to evaluate safety in delivering sequential laser energy from two different systems. The study was performed using subjects with skin types ranging from II-V (>50% V & IV-mean) and pigmented fine- and coarse-caliber hair. Each of the subjects underwent a series of laser treatments at monthly intervals and was treated with either the combination of 810 nm diode and 1,064 nm Q-switched Nd:YAG or the combination of the millisecond long pulsed 1,064 nm and the Q-switched short-pulse Nd:YAG lasers. Sequential treatments with two lasers were done during the same treatment session. A total of 22 anatomic sites were evaluated and compared using pre- and post-therapy pictures with magnification. The time of evaluation ranged from 3 to 48 months (a mean average of 12 months) following the last procedure. Each patient received laser therapy during the laser session with either a diode 810 nm or a LP:YAG laser followed by a QS:YAG laser, and the number of procedures ranged from 4 to 28 (13.5+/−6.7). In terms of ranges, a millisecond long pulse may have a pulse duration ranging from 0.1 milliseconds to 500 milliseconds, more preferably from 0.5 to 100 milliseconds or most preferably from 1 to 100 milliseconds. Similarly, a Q-switched short-pulse may have a pulse duration ranging from 1 femtosecond to 400 nanoseconds, or more preferably from 5 to 350 nanoseconds.

The study resulted in an increase in hair reduction compared to previous methods, and the treatments were well-tolerated. A combination of millisecond-long pulsed and Q-switched short-pulse lasers, yielding a 82% (+/−17%) hair reduction, has shown to be a more effective treatment option for laser hair removal, rather than the standard single laser treatment approach. Furthermore, aside from initial erythema and mild edema, no side effects were documented. Based on these and other results, there is an advantage in using various lasers for hair removal where both wavelength and pulse duration may be varied to enhance a particular outcome. For further information on hair removal using a Q-switched Nd:YAG laser, see Bakus, et al., “Long Term Fine Caliber Hair Removal With an Electro-Optic Q-Switched Nd:YAG Laser,” Lasers in Surgery and Medicine 42:706-711 (2010).

Lower energies also have an excellent safety profile exhibiting 45% reduction in hair counts at 6 months and greater than 50% reduction in hair counts at 24 months.

TABLE 2

| | | |
|---|---|---|
| % Hair Count | 3 Months | 6 Months |

| Reduction | SP | DP | SP | DP |
|---|---|---|---|---|
| Excellent | 29% | 29% | 17% | 50% |
| (>70%) | | | | |
| Moderate | 43% | 43% | 33% | 17% |
| (31-70%) | | | | |
| Fair | 29% | 29% | 50% | 33% |
| (<31%) | | | | |

The laser system disclosed in the present application may be operated in both a pulsed and a continuous mode, but preferably a combination of only millisecond-long and Q-switched short pulses. In this configuration, depending on the application, the millisecond-long and Q-switched, short-pulsed-laser energy may be delivered simultaneously or in succession. Using both approaches allows the clinician the ability to maximize the stimulatory and destructive advantages of both. Although the Nd:YAG laser may be used primarily in the following examples, any laser capable of being pulsed can be used, including at least those discussed above. Alternatively, two different laser types may be used in which one laser type is used in a millisecond-long pulse and the other in a Q-switched, short-pulse mode.

As seen in the following figures, treatment using both millisecond-long and Q-switched. short-pulse modes may be accomplished through the use of a combination laser system or the development of a single laser system capable of delivering different pulse durations (simultaneously or in succession). This may be accomplished by a laser system, for example, having the capability of producing both Q-switched and non-Q-switched pulse durations. As a result, multiple skin conditions (e.g., acne, skin aging and textural changes, pigmentary changes, scarring, and unwanted hair) may be treated using a single laser system. The successive application of these different pulse durations to the skin facilitates the improvement of the above conditions.

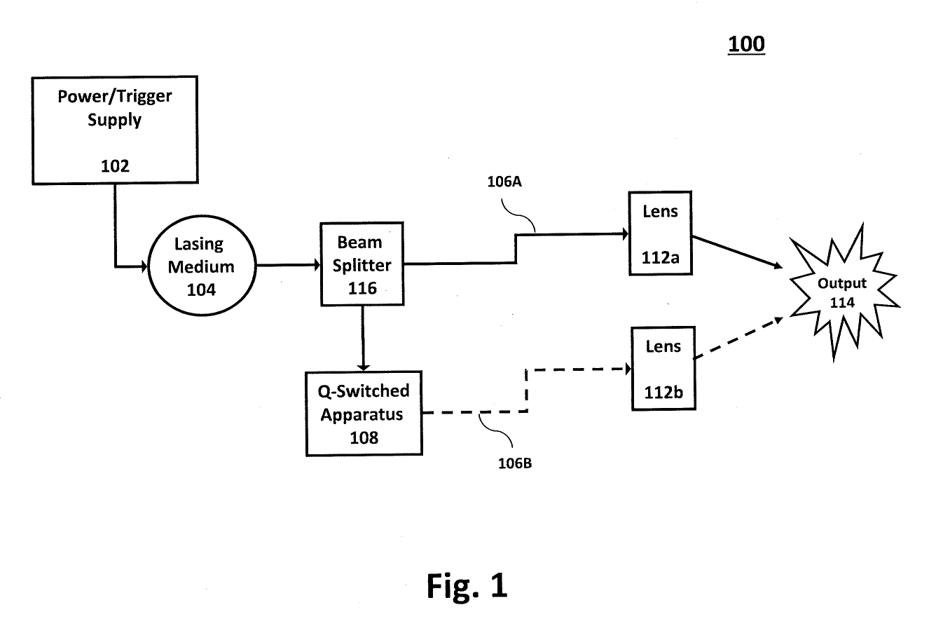

Referring now to FIG. 1, an exemplary high-level diagram of a first embodiment of a laser system 100 capable of emitting both millisecond-long and Q-switched short pulses is depicted. The laser system 100 may comprise a power/trigger supply 102, a lasing medium 104, one or more lenses 112a, 112b, a beam splitter 116, and a Q-switched apparatus 108. The power/trigger supply 102, or other external energy source, may provide any necessary power for laser pumping. Laser pumping may be achieved with electrical currents (e.g., semiconductors, or gases via high-voltage discharges) or with light, generated by discharge lamps or by other lasers (semiconductor lasers). However, a more exotic gain medium can be pumped by chemical reactions, nuclear fission, or with high-energy electron beams. While two lenses 112a, 112b are used in FIG. 1, a person having ordinary skill in the art would appreciate that additional lenses may be added or lenses may be removed to focus, adjust, and direct one or more laser beams (e.g., Beam A 106A and Beam B 106B) as desired by the physician or laser designer. Similarly, mirrors, fiber optics, and other light-bending devices may be used to aim and direct the one or more laser beams onto output 114.

In general, the lasing medium 104 is typically a source of optical gain within the laser system 100. The gain results from the stimulated emission of electronic or molecular transitions to a lower energy state from a higher energy state previously populated by a pump source. Examples of active laser media include, for example, the lasing mediums listed in Table 1, certain crystals, typically doped with rare-earth ions (e.g., neodymium, ytterbium, or erbium) or transition metal ions (titanium or chromium) most often yttrium aluminum garnet (YAG), yttrium orthovanadate (YVO₄), or sapphire (Al₂O₃); glasses, e.g., silicate or phosphate glasses, doped with laser-active ions; gases, e.g., mixtures of helium and neon (HeNe), nitrogen, argon, carbon monoxide, carbon dioxide, or metal vapors; semiconductors, e.g., gallium arsenide (GaAs), indium gallium arsenide (InGaAs), or gallium nitride (GaN); and liquids, in the form of dye solutions as used in dye lasers. For further information on laser dye wavelengths and their uses, see, for example, http://www.exciton.com/wavelength_chart.html and Jung et al., “Comparison of a Pulsed Dye Laser and a Combined 585/1064-nm Laser in the Treatment of Acne Vulgaris”, Dermatol Surg 2009; 35:1181-1187.

As mentioned, a laser beam generated by the power/trigger supply 102 and lasing medium 104 may be adjusted, or focused, using one or more lenses. However, the laser beam may also be split into two separate beams using a beam splitter 116—an optical device used to split a beam of light into two or more beams. A common type of beam splitter 116 may be a cube made from two triangular glass prisms glued together at their base using, for example, Canada balsam (e.g., turpentine made from the resin of the balsam fir tree, Abies balsamea). The thickness of the resin layer may be adjusted such that (for a known wavelength) half of the light incident through one port (i.e., face of the cube) may be reflected and the other half may be transmitted due to frustrated total internal reflection. Polarizing beam splitters, such as the Wollaston prism, use birefringent materials, splitting light into beams of differing polarization.

Another suitable beam splitter 116 may be constructed using a half-silvered mirror (e.g., a glass plate with a thin aluminum coating) where the thickness of the coating may be such that part, typically half, of light incident at a 45-degree angle may be transmitted, and the remainder reflected. Instead of a metallic coating, a dielectric optical coating may be used. Such mirrors are commonly used as output couplers in laser construction. Depending on the coating being used, reflection/transmission ratios may differ in function of the wavelength. A third beam splitter design may be a dichroic mirrored prism assembly that uses dichroic optical coatings to divide an incoming light beam into a number of spectrally distinct output beams.

Once the beam has been split into Beam A 106A and Beam B 106B using the beam splitter 116, Beam A may proceed directly to lens 112a where it may be focused, or otherwise adjusted, to hit output target 114. The output target 114 would typically be the area being treated by the laser (e.g., a patient's skin or other body part). However, contrary to Beam A 106A, Beam B 106B is diverted to a Q-switching apparatus 108 prior to lens 112b.

The Q-switching apparatus 108 may employ either passive or active switching techniques. In passive Q-switching, the Q-switch may use a saturable absorber—a material whose transmission increases when the intensity of light exceeds some threshold. The material may be, for instance, an ion-doped crystal like Cr:YAG, which can be used for Q-switching of Nd:YAG lasers, a bleachable dye, a passive semiconductor device, or the like. In active Q-switching, the Q-switch may be a mechanical device such as a shutter, chopper wheel, or spinning mirror/prism placed inside the cavity, or some form of modulator such as an acousto-optic device or an electro-optic device (e.g., a Pockels cell or Kerr cell). The reduction of losses (increase of Q) may be triggered by an external event, typically an electrical signal; thus pulse repetition rate can be externally controlled. The Q-switched Beam B may then proceed to lens 112b where it may be focused, or otherwise adjusted, to hit an output target 114. Exemplary Q-switching systems and techniques may include, for example: U.S. Pat. No. 5,018,152 to Linne et al., entitled “Apparatus for controlling pulse energy in a Q-switched laser system;” U.S. Pat. No. 5,394,413 to Zayhowski, entitled “Passively Q-switched picosecond microlaser;” U.S. Pat. No. 5,905,746 to Nguyen, entitled “Q-switch laser method and apparatus;” and U.S. Pat. No. 7,649,920 to Welford, entitled “Q-switched microlaser apparatus and method for use.”

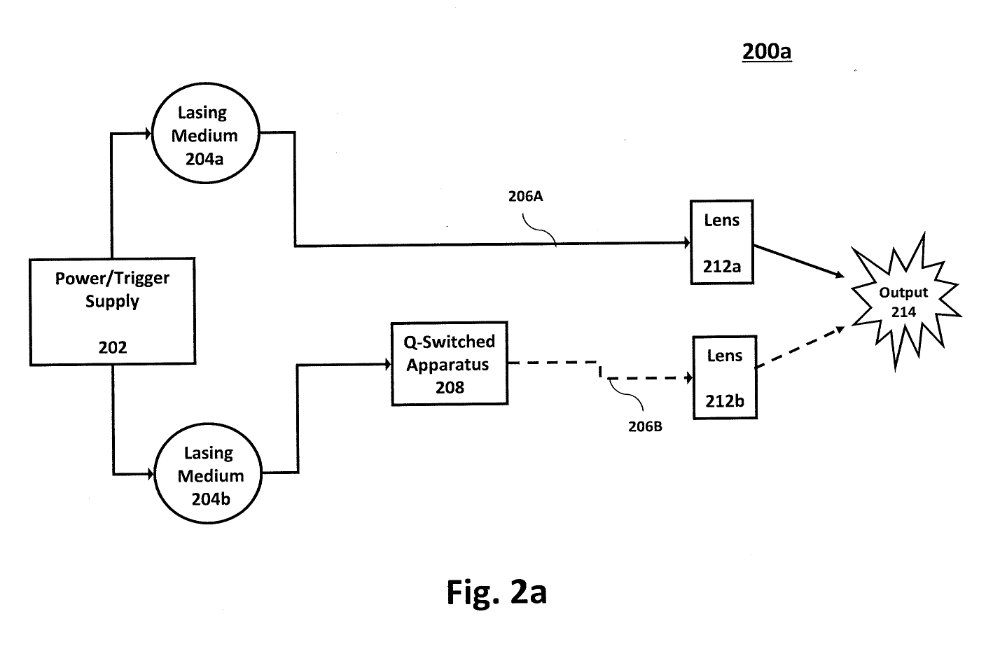

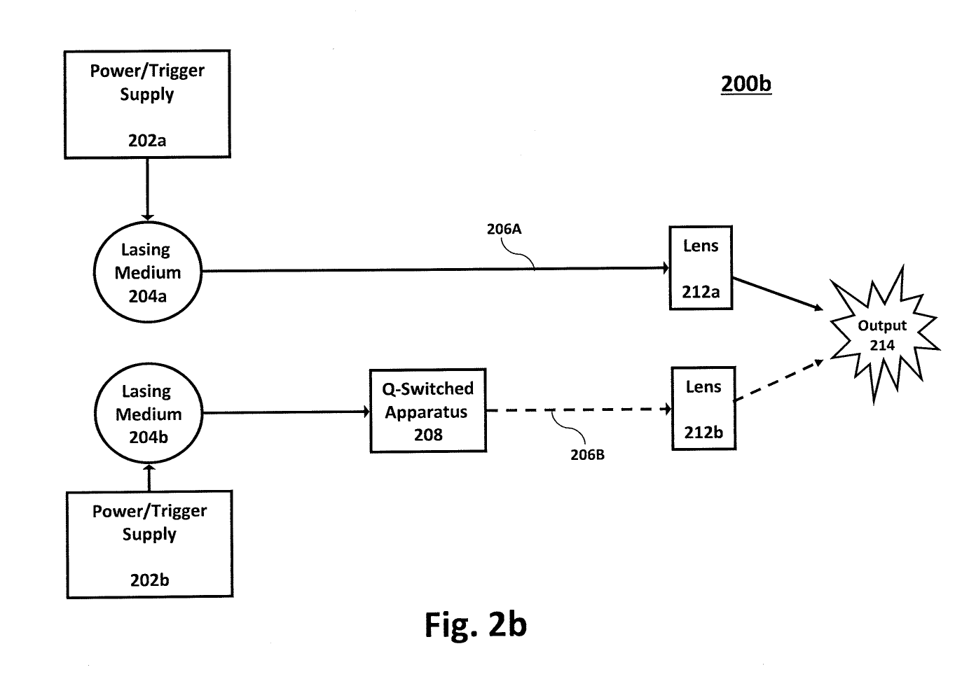

Now referring to FIG. 2a, an exemplary high-level diagram of a second embodiment of a laser system 200a enabled to emit both millisecond-long and Q-switched short pulses is shown. The laser system 200a generally comprises a power/trigger supply 202, lasing mediums 204a, 204b, one or more lenses 212a, 212b, and a Q-switched apparatus 208. The individual components function similarly to those of FIG. 1 except, rather than using a beam splitter to generate two beams, two lasing mediums 204a, 204b are employed. Removal of the beam splitter eliminates any beam intensity reduction potentially caused by the beam splitter. Furthermore, by implementing two lasing mediums, a designer may use one lasing medium for the Q-switched short-pulse laser beam and a second lasing medium for the millisecond-long pulses to achieve a desired combination of wavelengths/pulse speeds.

While FIGS. 1 through 2a illustrate systems with a single power/trigger supply, as seen in FIG. 2b, millisecond-long and Q-switched short pulses may be delivered using two separate systems or subsystems. An exemplary embodiment of a laser system 200b enabled to emit both millisecond-long and Q-switched short pulses is shown in FIG. 2b. The laser system 200b generally comprises power/trigger supplies 202a, 202b, lasing mediums 204a, 204b, one or more lenses 212a, 212b, and a Q-switched apparatus 208. The individual components function similarly to those of FIG. 2b except, rather than using a single system having a single power/trigger supply, two power/trigger supplies 202a, 202b are employed such that the long and Q-switched laser subsystems are independent of one another. For example, a physician may choose to treat a patient using two separate systems (or devices/apparatuses) where one system is enabled to output millisecond-long and the other is enabled to output Q-switched short pulses. Accordingly, a first power supply device, a first lasing medium, and the Q-switched apparatus may be contained within a first housing configured to output Q-switched short pulsed laser energy while a second power supply device and second lasing medium may be contained within a second housing and configured to output millisecond long pulsed laser energy. Alternatively, a power supply device, a lasing medium, and Q-switched apparatus may be contained within a single housing and configured to output both millisecond long pulsed laser energy and Q-switched short pulsed laser energy. As one of ordinary skill in the art would appreciate, there are numerous ways to arrange a system or apparatus enabled to deliver millisecond-long and the Q-switched short pulses. Even in instances where two separate systems or apparatuses are used to output millisecond-long and the Q-switched short pulses, the two systems may be installed or situated in a common housing, wheeled cart, or apparatus.

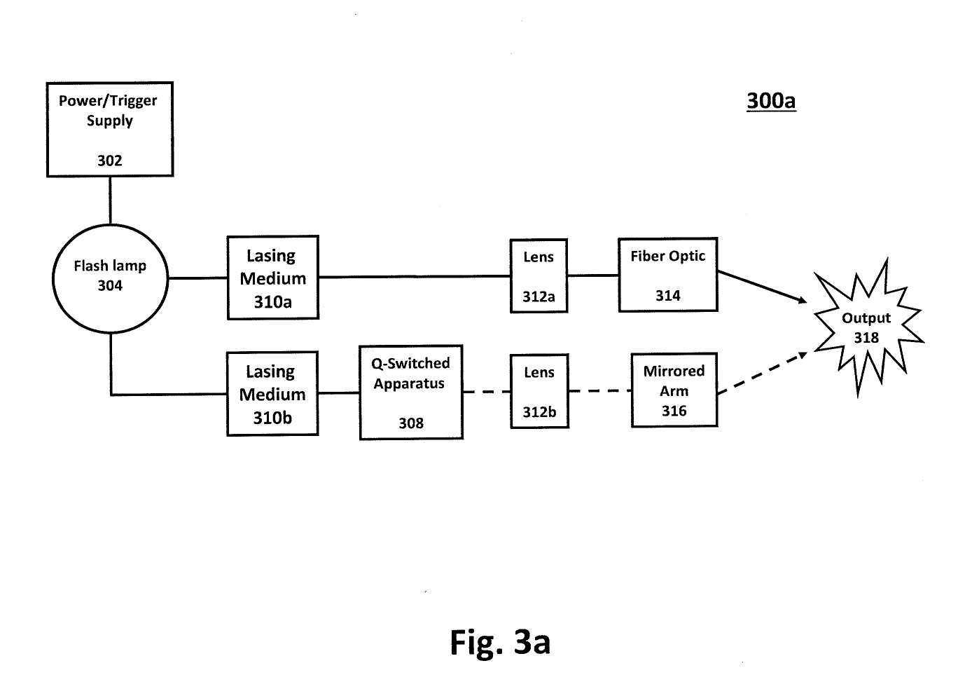

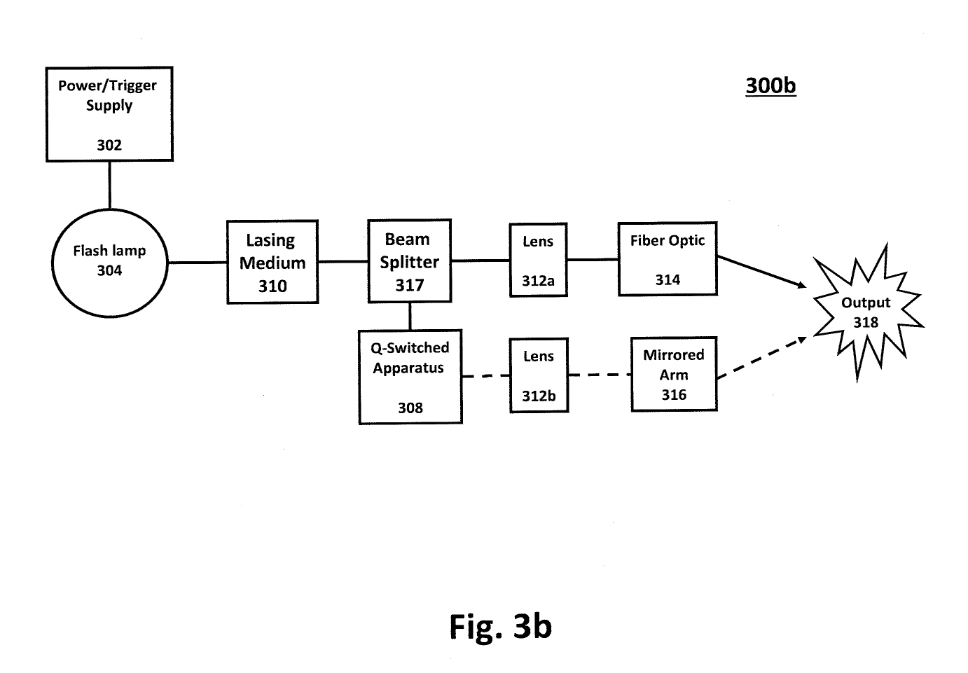

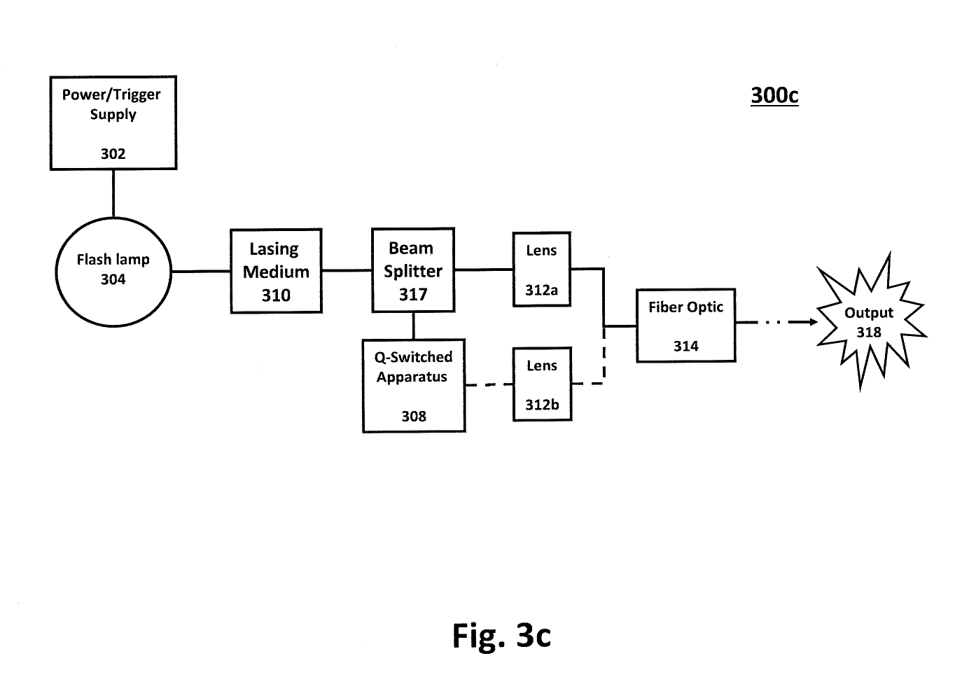

FIG. 3a illustrates an exemplary high-level diagram of a third embodiment of a laser 300a enabled to emit both millisecond-long and Q-switched short pulses is shown. The laser system 300a generally comprises a power/trigger supply 302, a flash lamp 304, lasing mediums 310a, 310b, fiber optics 314, a mirrored arm 316, one or more lenses 312a, 312b, and a Q-switching apparatus 308. As in the previous examples, the output target 318 would be the area being treated by the laser (e.g., a patient's skin or other body part).

The flash lamp 304 may be an electric glow discharge lamp designed to produce extremely intense, incoherent, full-spectrum white light for very short durations. Flashtubes are often made of a length of glass tubing with electrodes at either end and are filled with a gas that, when triggered, ionizes and conducts a high-voltage pulse to produce the light. While flashtubes are often used for photographic purposes, they are also employed in scientific, medical, and industrial applications, including lasers. Flash lamps are often used in conjunction with dye lasing medium yielding a number of wavelengths, including, for example, 390-435 nm (stilbene), 460-515 nm (coumarin 102), 570-640 nm (rhodamine 6G), and many others. Nevertheless, a flash lamp may be used with ruby, Nd:YAG, Er:YAG, and countless other lasing mediums. While a single type of lasing medium may be used to generate both the millisecond-long and Q-switched, short-pulse laser beams, a designer should not be restricted to a single lasing medium and, to achieve a desired wavelength, may use one lasing medium for the Q-switched, short-pulse laser beam and a second lasing medium for millisecond-long pulses.

Now referring to FIG. 3b, a high-level diagram of a fourth embodiment of a laser system 300b enabled to emit both millisecond-long and Q-switched short pulses is shown. The laser system 300b generally comprises a power/trigger supply 302, a lasing medium 310, one or more lenses 312a, 312b, a Q-switched apparatus 308, and a beam splitter 317. The individual components function similarly to those of FIG. 3a except, rather than using two lasing mediums 310a, 310b, a beam splitter 317 may be used to generate two beams. While the laser systems of FIGS. 3a and 3b illustrate the millisecond-long pulse being delivered using a fiber optic delivery system and the q-switched being delivered using a mirrored arm, the delivery methods may be swapped such that the millisecond-long pulse is delivered using a mirrored arm and the q-switched is delivered using a fiber optic delivery system. Similarly, as depicted in FIG. 3c, the millisecond-long and q-switched pulses may be delivered through a single delivery system (e.g., both via fiber optic). While a fiber optic 314 delivery system is depicted in FIG. 3c, other comparable deliver systems may be used.

While FIGS. 1 through 3 illustrate exemplary laser systems, a person having ordinary skill in the art would appreciate that other laser configurations and arrangements may be devised to output combination millisecond-long and Q-switched short-pulse laser exposures. Therefore, the present application should not be considered limited to the configurations shown in FIGS. 1 through 3. For example, Aerolase offers exemplary lasers and laser devices, including hand held laser device, that may be configured to carry out the present invention. For further information on Aerolase's technology, see, for example, U.S. Pat. Nos. 5,868,731; 6,251,102; 6,313,289; 6,733,493; 6,813,289, 7,298,767, 7,524,317, 7,548,568 and US Patent Pub. No. 2009/0257465. Additional information can be found on their website, available at www.aerolase.com.

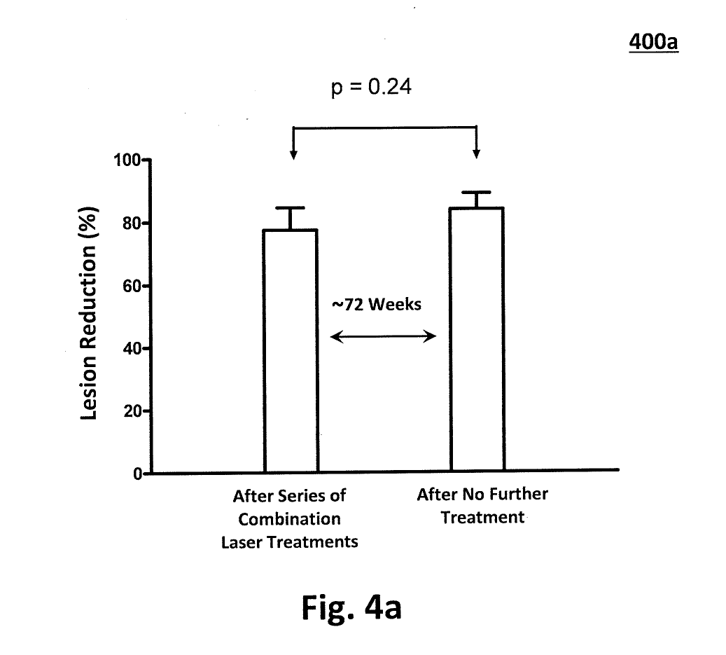

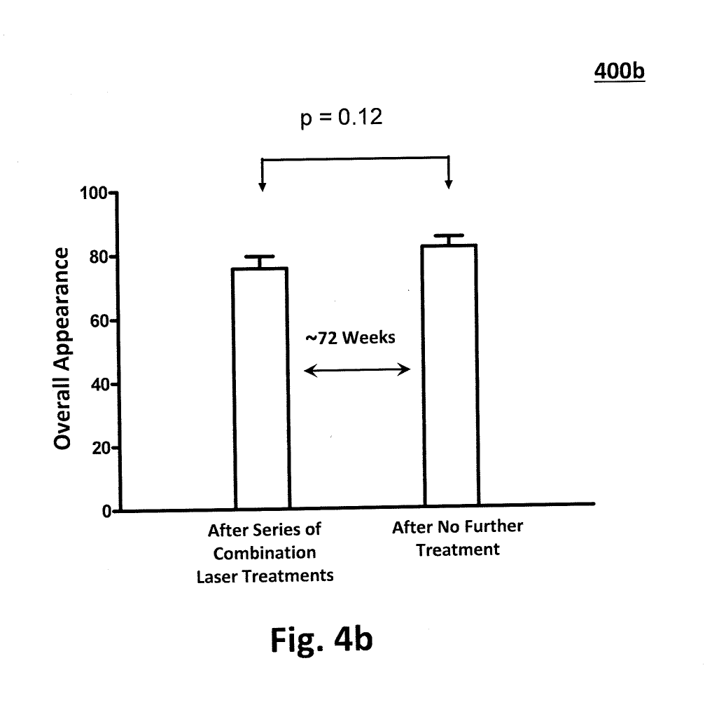

Referring now to FIGS. 4a and 4b, the figures illustrate results observed during experimental treatments using a combination millisecond-long and Q-switched short-pulse laser exposures. During the study, thirteen subjects suffering from acne underwent combination laser treatments using a millisecond long pulse and a Q-switched short pulse ND:YAG systems. The average age of the subjects was 25 years (ranging from 18-37 years old) and the average pretreatment acne severity score was 4.5 on a scale of 1 to 5 (5 being most severe). The series of combination laser treatments comprised of about four to eight laser treatment sessions performed at 2 to 4 week intervals where each subject's level of improvement was evaluated by two independent blinded evaluators. During the study, the millisecond long pulse duration was set to 60 milliseconds while the Q-switched short-pulse duration ranged from 5 to 10 nanoseconds.

FIG. 4a is bar graph comparing the percentage of a skin area's lesion reduction observed in a first evaluation, performed shortly after a series of combination laser treatments (4-8 treatments, each being 2-4 weeks apart), and a second evaluation, performed 7 to 139 weeks (mean average of 72 weeks) after the last treatment. As seen in FIG. 4a, shortly after the series of treatments, patients exhibited lesion reduction of 78%. By the second evaluation, having received no further treatments, the patients' skin continued to improve, exhibiting a lesion reduction of 84%. These results clearly demonstrate that when patients are treated using a combination of both millisecond-long and Q-switched short pulses, their skin continues to improve while not requiring ongoing or subsequent treatment.

Similarly, FIG. 4b is a bar graph comparing the overall skin appearance after both the first and second evaluation. As seen in FIG. 4b, shortly after the series of treatments, patients exhibited an improved skin appearance of about 78%. By the second evaluation, having received no further treatment, the patient's skin continued to maintain improvement, exhibiting improved skin appearance of about 80%. For the study, the patients' skin was evaluated by two independent blinded evaluators using factors such as skin texture, scarring, pore size, and other observed defects.

Therefore, as illustrated in both FIGS. 4a and 4b, patients exhibited improvements in both the percentage of acne lesion reduction and overall skin appearance even months after the final combination laser treatment.

Because a notable parameter in laser treatment is the pulse duration, the time of laser emission irradiation, limiting the pulse duration of the laser exposure limits the effects on the target. Various biologic effects occur within exposed tissue depending on the length of laser exposure. For example, pulse durations that are in the millisecond range produce thermal changes to the tissue, while those in the nanosecond range also contribute to acoustic tissue effects. Laser pulses stimulate collagen production and can also be used to improve the aforementioned conditions.

Until now, the approach by medicine and surgery has been to treat tissue using either millisecond-long or Q-switched, short-pulse exposure. As exemplified by the foregoing, using a combination of both millisecond-long and Q-switched short-pulse exposures during therapy allows for improved tissue effects. The laser emissions can be presented with varying time intervals depending on the desired final tissue outcome. Longer pulses can range from less than a millisecond to more than 500 milliseconds, while the Q-switched system can produce pulses ranging from femtoseconds to nanoseconds. More specifically, a millisecond long pulse may have a pulse duration ranging from 0.1 milliseconds to 500 milliseconds, more preferably from 0.5 to 100 milliseconds or most preferably from 1 to 100 milliseconds. Conversely, a Q-switched short-pulse may have a pulse duration ranging from 1 femtosecond to 400 nanoseconds or more preferably from 5 to 350 nanoseconds. However, the length of the pulses may be adjusted to accommodate a particular patient's skin condition or skin-type.

As discussed above and illustrated in the Figures, using both millisecond-long and Q-switched, short-pulsed lasers (e.g., Nd:YAG) in a single treatment of multiple cutaneous conditions produces outcomes far superior to those achieved by either modality alone. This laser configuration can be used for the treatment of skin changes caused by aging, skin texture irregularities, enlarged pores, scars, pigmentary changes such as decrease or increase of pigment, diminishment of luster, acne, excessive sebum (oil) production, sebaceous hyperplasia (enlarged oil glands) and unwanted hair. In fact, this novel approach may also be used to treat other diseases, including various inflammatory skin conditions, skin growths, cancers and conditions in other parts of the body. For example, kidney stones, vessel plaque, and gallbladder stones may be treated by applying millisecond-long and Q-switched short laser pulses using fiber optics and a catheter. Using this treatment for kidney stones, often referred to as laser lithotripsy, involves inserting a small instrument into the urethra and snaking it into the ureter containing the stone. Millisecond-long and Q-switched short combination laser energy may then be used to slowly break the stone into fragments without harming the surrounding tissues and muscles, using, for instance, a fiber optic cable.

Although various embodiments have been described with reference to a particular arrangement of parts, features, and the like, these are not intended to exhaust all possible arrangements or features, and indeed many other embodiments, modifications, and variations will be ascertainable to those of skill in the art. All U.S. and foreign patent documents, and all articles, brochures, and other published documents discussed above are hereby incorporated by reference into the Detailed Description.

What is claimed is:

1. A method for treating a condition, comprising:
administering a Q-switched short pulsed ND:YAG laser energy having a pulse duration between 5 and 350 nanoseconds; and
administering a millisecond long pulsed ND:YAG laser energy having a pulse duration between 1 and 100 milliseconds;
wherein the Q-switched short pulsed laser energy and millisecond long pulsed laser energy are both applied to treat a health or age related condition.

2. The method of claim 1, wherein the health or age related condition is a skin condition chosen from a group consisting of: inflammatory skin conditions, acne, skin aging, skin laxity, skin texture irregularities, enlarged pores, scars, pigmentary changes, changed skin color, skin luster, skin growths, excessive sebum (oil) production, sebaceous hyperplasia (enlarged oil glands), cancers, striae (stretch marks) and combinations thereof.

3. The method of claim 1, wherein the health or age related condition is chosen from a group consisting of: kidney stones, gallbladder stones, vessel plaque, and combinations thereof.

4. The method of claim 1, wherein the Q-switched short pulsed laser energy and millisecond long pulsed laser energy are delivered simultaneously.

5. The method of claim 1, wherein the Q-switched short pulsed laser energy and millisecond long pulsed laser energy are delivered using a single laser system.

6. The method of claim 1, wherein the Q-switched short pulsed laser energy and millisecond long pulsed laser energy are delivered from a single device enabled to produce both Q-switched short and millisecond long pulsed energies.

7. A method for treating a skin condition, comprising:
administering a Q-switched short pulsed laser energy; and
administering a millisecond long pulsed laser energy;
wherein the Q-switched short pulsed laser energy and millisecond long pulsed laser energy are both applied to treat a skin condition.

8. The method of claim 7, wherein the Q-switched short pulsed laser energy has a pulse duration between 0.1 and 500 milliseconds and the Q-switched short-pulse has a pulse duration between 1 femtosecond and 400 nanoseconds.

9. The method of claim 7, wherein the Q-switched short pulsed laser energy has a pulse duration between 1 and 100 milliseconds and the Q-switched short-pulse has a pulse duration between 5 to 350 nanoseconds.

10. The method of claim 7, wherein the Q-switched short pulsed laser energy and millisecond long pulsed laser energy are delivered in a successive manner starting with either pulse.

11. The method of claim 7, wherein the Q-switched short pulsed laser energy and millisecond long pulsed laser energy are delivered simultaneously.

12. The method of claim 7, wherein the Q-switched short pulsed laser energy and millisecond long pulsed laser energy are delivered using a single laser system.

13. The method of claim 7, wherein the Q-switched short pulsed laser energy and millisecond long pulsed laser energy are delivered from a single device enabled to produce both Q-switched short and millisecond long pulsed energies.

14. The method of claim 7, wherein the millisecond long pulsed laser energy or Q-switched short pulsed laser energy are generated using a lasing medium chosen from a group consisting of: ruby crystal, Nd:YAG, alexandrite, DPSS (diode pumped solid state), Nd:YVO₄ (yttrium orthovanadate), Nd:YLF (yttrium lithium fluoride), Er:YAG, thulium, argon, dye, copper, gold vapor, krypton, KTP, diode, Er:glass, Er:YSGG, intense pulse light, holmium, CO₂, and combinations thereof.

15. The method of claim 7, wherein the millisecond long pulsed laser energy and Q-switched short pulsed laser energy use a single beam-delivery system.

16. The method of claim 7, wherein the method is used to treat a skin condition chosen from group consisting of: inflammatory skin conditions, acne, skin aging, skin laxity, skin texture irregularities, enlarged pores, scars, pigmentary changes, changed skin color, skin luster, skin growths, excessive sebum (oil) production, sebaceous hyperplasia (enlarged oil glands), cancers, striae (stretch marks) and combinations thereof.

17. The method of claim 7, wherein the method is used for hair or tattoo removal.

18. A laser system for treating one or more conditions, comprising:
a power supply device;
a lasing medium;
one or more lenses; and
a Q-switched apparatus for outputting Q-switched short pulsed laser energy;
wherein the laser system is configured to output both millisecond long pulsed laser energy having a pulse duration between 0.1 and 500 milliseconds and Q-switched short pulsed laser energy having a pulse duration between 1 femtosecond and 400 nanoseconds.

19. The laser system of claim 18, wherein the power supply device, lasing medium, and Q-switched apparatus are contained within a single housing configured to output both millisecond long pulsed laser energy and Q-switched short pulsed laser energy.

20. The laser system of claim 18, wherein a first power supply device, a first lasing medium, and the Q-switched apparatus are contained within a first housing configured to output Q-switched short pulsed laser energy and a second power supply device and second lasing medium are contained within a second housing configured to output millisecond long pulsed laser energy.

21. The laser system of claim 18, wherein the laser system delivers Q-switched short pulsed laser energy and millisecond long pulsed laser energy in a successive manner starting with either pulse.

22. The laser system of claim 18, wherein the laser system delivers Q-switched short pulsed laser energy and millisecond long pulsed laser energy simultaneously.

23. The laser system of claim 18, wherein the lasing medium is chosen from a group consisting of: ruby crystal, Nd:YAG, alexandrite, DPSS (diode pumped solid state), Nd:YVO₄ (yttrium orthovanadate), Nd:YLF (yttrium lithium fluoride), Er:YAG, thulium, argon, dye, copper, gold vapor, krypton, KTP, diode, Er:glass, Er:YSGG, intense pulse light, holmium, CO₂ and combinations thereof.

24. The laser system of claim 18, wherein the millisecond long pulsed laser energy and Q-switched short pulsed laser energy use a single beam-delivery system.

25. The laser system of claim 18, wherein the condition is a skin condition chosen from a group consisting of: inflammatory skin conditions, acne, skin aging, skin laxity, skin texture irregularities, enlarged pores, scars, rosacea, striae, redness, pigmentary changes, changed skin color, skin luster, skin growths, excessive sebum (oil) production, sebaceous hyperplasia (enlarged oil glands), cancers, striae (stretch marks) and combinations thereof.

26. The laser system of claim 18, wherein the condition is chosen from group consisting of: kidney stones, gallbladder stones, vessel plaque, and combinations thereof.

27. The laser system of claim 18, wherein the laser system is used for hair or tattoo removal.

28. A laser apparatus for treating one or more skin conditions comprising:
a power supply device;
a lasing medium;
one or more lenses; and
a Q-switched apparatus for outputting Q-switched short pulsed laser energy;
wherein the laser system is used to treat a skin condition and is configured to output both millisecond long pulsed laser energy having a pulse duration between 0.1 and 500 milliseconds and Q-switched short pulsed laser energy having a pulse duration between 1 femtosecond and 400 nanoseconds.

29. The laser apparatus of claim 28, wherein the lasing medium is chosen from a group consisting of: ruby crystal, Nd:YAG, alexandrite, DPSS (diode pumped solid state), Nd:YVO₄ (yttrium orthovanadate), Nd:YLF (yttrium lithium fluoride), Er:YAG, thulium, argon, dye, copper, gold vapor, krypton, KTP, diode, Er:glass, Er:YSGG, intense pulse light, holmium, CO₂ and combinations thereof.

30. The laser system of claim 28, wherein the laser system delivers Q-switched short pulsed laser energy and millisecond long pulsed laser energy in a successive manner starting with either pulse.

31. The laser system of claim 28, wherein the laser system delivers Q-switched short pulsed laser energy and millisecond long pulsed laser energy simultaneously.

32. A method for treating a skin condition, comprising:
using a first laser system to administer a Q-switched short pulsed laser energy; and
using a second laser system to administer a millisecond long pulsed laser energy;
wherein the Q-switched short pulsed laser energy and millisecond long pulsed laser energy are applied to treat a skin condition.

33. The method of claim 32, wherein the Q-switched short pulsed laser energy has a pulse duration between 0.1 and 500 milliseconds and the Q-switched short-pulse has a pulse duration between 1 femtosecond and 400 nanoseconds.

34. The method of claim 32, wherein the Q-switched short pulsed laser energy has a pulse duration between 1 and 100 milliseconds and the Q-switched short-pulse has a pulse duration between 5 to 350 nanoseconds.
